(19) United States
(12) Patent Application Publication
(10) Pub. No.: US 2005/0049666 A1
(43) Pub. Date: Mar. 3, 2005

(54) Stent delivery system

(76) Inventors: Thomas Yung-Hui Chien, San Jose, CA (US); Huey Quoc Chan, San Jose, CA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/648,459

(22) Filed: Aug. 26, 2003

(51) Int. Cl. A61F 2/06

(52) U.S. Cl. 623/1.11

(57) ABSTRACT

A stent delivery system is disclosed which includes an inner tube disposed within an outer tube in an annular space disposed therebetween. The distal end of the inner tube extends beyond the distal end of the outer tube and a balloon extends between the distal end of the outer tube and the distal end of the inner tube. A stent is disposed over the balloon. A heating mechanism is provided to heat the balloon which, in turn, transmits heat to the stent for expansion. An inflation media for the balloon is transported through the annular space to ensure continuous contact between the balloon and stent during deployment of the stent.

### BACKGROUND

1. Technical Field

A stent delivery system is disclosed wherein a shape memory stent is expanded with heat.

2. Description of the Related Art

Intravascular catheters are widely used for a variety of diagnostic and therapeutic purposes. Specifically, angioplasty has been developed as an alternative to bypass surgery for treating vascular diseases or other conditions that occlude or reduce blood flow in a patient's vascular system. Balloon angioplasty has proven to be a useful and often a preferred treatment for coronary diseases that cause blockages, also known as stenosis, in coronary arteries as well as other parts of the vascular system.

One current angioplasty technique makes use of a single operator exchange (SOE) or rapid exchange catheter assembly as illustrated in U.S. Pat. No. 5,156,594. The catheter assembly shown therein includes a balloon catheter having a proximal end including a hypotube. A medial shaft segment and a more flexible distal end portion has a balloon mounted radially proximate its distal end. The balloon is in fluid communication with the hypotube. The balloon catheter includes a main lumen that is in communication with the lumen of the hypotube as well as a relatively short separate lumen which accommodates a guidewire.

In practice, when using an over the wire (OTW) or SOE catheter, a guidewire is inserted into the patient's vascular system through a guide catheter. The balloon catheter assembly is then fed through the guide catheter and over the guidewire. The stent delivery or balloon catheter is loaded onto the guidewire by inserting the proximal end of the guidewire into the very distal end of the catheter. While maintaining the position of the guidewire within the vascular system, the catheter is advanced along the length of the guidewire. The proximal end of the guidewire may exit the proximal guide wire port of the catheter. With a SOE catheter, the guidewire typically exits proximal to the balloon, within the distal third of the length of the catheter. In the case of an OTW, the guidewire traverses the full length of the catheter and exits the manifold at the proximal end of the catheter.

Advancing the catheter assembly to position the balloon across a stenosis can be a difficult and time consuming task due to the narrow and tortuous passages through which the catheter assembly must be passed. The balloon must be positioned precisely and movement of the balloon through the vascular system must be conducted in as atraumatic manner as possible.

However, while angioplasty is effective in alleviating arterial stenosis in an artery or vessel, in many cases, the vessel may restenose or close down, thereby negating the positive affect of the angioplasty procedure, and possibly requiring an additional angioplasty procedure. To reduce the risk of restenosis, various stent devices have been used for mechanically keeping the affected vessel area open after completion of the angioplasty procedure.

Self-expanding and balloon expandable stents are known. Expandable stents generally are conveyed to the lesion to be treated on a balloon catheter or other expandable device. The stent is positioned within the vasculature system in a compressed configuration along the balloon catheter which is typically folded or wrapped to make the diameter of the balloon catheter and the stent as small as possible. After the stent is positioned across the lesion, the balloon and stent are expanded using pressure conveyed to the interior of the balloon through the catheter. However, self expanding stents typically require an additional protective sheath over the outside of the balloon catheter and stent to prevent the stent from prematurely expanding as the balloon catheter and stent proceed up through the vasculature system to the lesion to be treated. The additional protective sheath enlarges the cross-sectional diameter of the stent delivery device and reduces the flexibility and therefore the trackability of the device.

Self expanding and balloon expandable stents are known. Balloon expandable stents are generally crimped down onto the balloon for delivery within the vascular system.

Self-expanding stents can be expanded using various mechanical means such as pistons, sleeves or wires. Self- expanding stents may be maintained in their radially reduced delivery state by a number of means, including a sheath. The sheath is advanced either proximally or distally along the axial length of the catheter to expose the stent, allowing the stent to deploy. Also, self-expanding stent delivery systems may also employ water-soluble retaining bands or rings. When exposed to blood, the water-soluble retaining rings dissolve thereby allowing the stent to expand. Like the pressure-expandable stents, self-expanding stents also must be protected with an additional sheath. Self expanding stent delivery systems that employ the use of a sheath or other means to fix the stent to the delivery catheter may be less flexible and detract from the trackability and general handling of the stent delivery device.

Accordingly, as angioplasty and stent delivery procedures continue to increase, there is a need to provide improved stent delivery systems with improved trackability and flexibility.

### SUMMARY OF THE DISCLOSURE

In satisfaction of the aforenoted needs, a stent delivery system is disclosed which comprises an inner tube comprising a proximal end and a distal end. The inner tube is disposed within an outer tube with an annular space disposed therebetween. The distal end of the inner tube further comprises a distal tip. The outer tube comprises a proximal end and a distal end. The distal end of the outer tube is disposed proximally to the distal tip of the inner tube. The distal end of the outer tube is connected to a balloon which extends between the distal end of the outer tube and the distal tip of the inner tube. The balloon is designed to be disposed within and engage a cylindrical expandable stent which overlies the balloon and is disposed between the distal end of the outer tube and the distal tip of the inner tube. The distal tip of the inner tube may have a maximum outer diameter that is equal to or greater than a maximum outer diameter of the stent in its unexpanded form to protect the stent during movement of the stent through the patient's vascular system.

The balloon is preferably heated which, in turn, heats the expandable stent which, in turn, results in expansion of the stent. The balloon may be heated by flowing the heated medium through the annular space to the interior of the balloon. In addition, a heating element may be disposed between the balloon and the distal end of the inner tube which can be used to heat the balloon and, in turn, the stent, for expansion thereof.

A method of deploying an expandable stent in a vasculature system is also disclosed which comprises providing a stent delivery system as described above, inserting the stent delivery system, with the stent in the unexpanded form, into the vasculature system to a desired position, heating at least one of the balloon and the stent while at least partially inflating the balloon to expand the stent and causing it to adhere to against the vasculature system vascular wall at the desired position. Subsequently deflating the balloon, and withdrawing the inner and outer tubes and balloon delivery device from the vasculature system.

### BRIEF DESCRIPTION OF THE DRAWINGS

The disclosed devices and methods are described more or less diagrammatically in the accompanying drawings wherein:

FIG. 1 is a partial sectional view of a disclosed stent delivery device illustrating the distal end of the inner tube and heating element;

FIG. 2 is a partial sectional view of the distal end of a disclosed stent delivery device further illustrating the distal end of the outer tube and balloon extending between the outer tube and the distal tip of the inner tube;

FIG. 3 is another partial sectional view of a disclosed stent delivery device further illustrating placement of an expandable stent over the balloon;

FIG. 4 is a partial sectional view of a disclosed stent delivery device as extended over a guidewire within a vasculature system;

FIG. 5 is a partial sectional view of the stent delivery device shown in FIG. 4 after partial inflation of the balloon and partial expansion of the stent within the area of the vasculature system to be treated;

FIG. 6 is a partial section view of the stent delivery device shown in FIGS. 4 and 5 after full expansion of the stent and further expansion of the balloon; and

FIG. 7 is another partial sectional view of the stent delivery device shown in FIGS. 4-6 after deflation of the balloon and prior to removal of the delivery device from the vasculature system leaving the stent in place along the area to be treated as shown.

Although the above-identified figures disclose a single embodiment, other variations thereof are also contemplated. It should be understood, that numerous modifications and other alternative embodiments can be devised by those skilled in the art after reading this disclosure which will fall within the spirit and scope of this disclosure and the appended claims.

### DETAILED DESCRIPTION OF THE ILLUSTRATED EMBODIMENTS

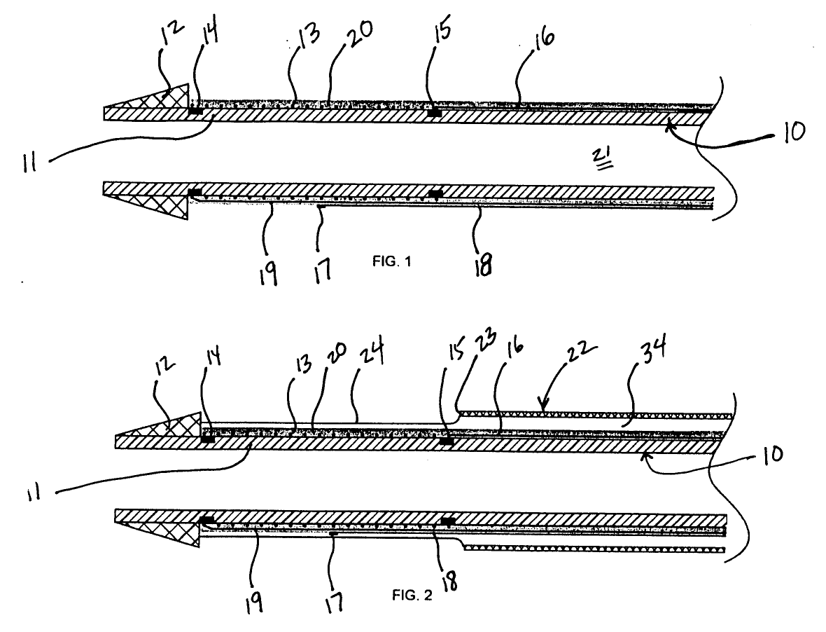

One embodiment of the disclosed stent delivery device is illustrated in FIGS. 1-7. Turning to FIG. 1, an inner tube 10 is provided having a distal end 11 connected to a tapered distal tip 12. In the embodiment illustrated in FIG. 1, a heating element 13 is positioned around the distal end 11 of the inner tube 10. The heating element 13 is connected to a lead wire 16 and a return wire 19 to provide current thereto. The distal end 11 of the inner tube 10 can be equipped with a distal radiopaque marker 14 and a proximal radiopaque marker 15. As shown below, the radiopaque markers 14, 15 can be used to indicate the location of the distal and proximal ends of a stent for purposes of positioning the stent for expansion. A thermocouple 17 may also disposed on the distal end 11 of the inner tube 10 and can be connected to a lead wire 18 for indicating balloon temperature to the physician. An insulating layer 20 may be employed to prevent leakage and shorting of the circuits. The wires 16, 18 and 19 may also be individually insulated. The inner tube 10 provides a lumen 21 for accommodating a guidewire 27 (see FIGS. 4-7).

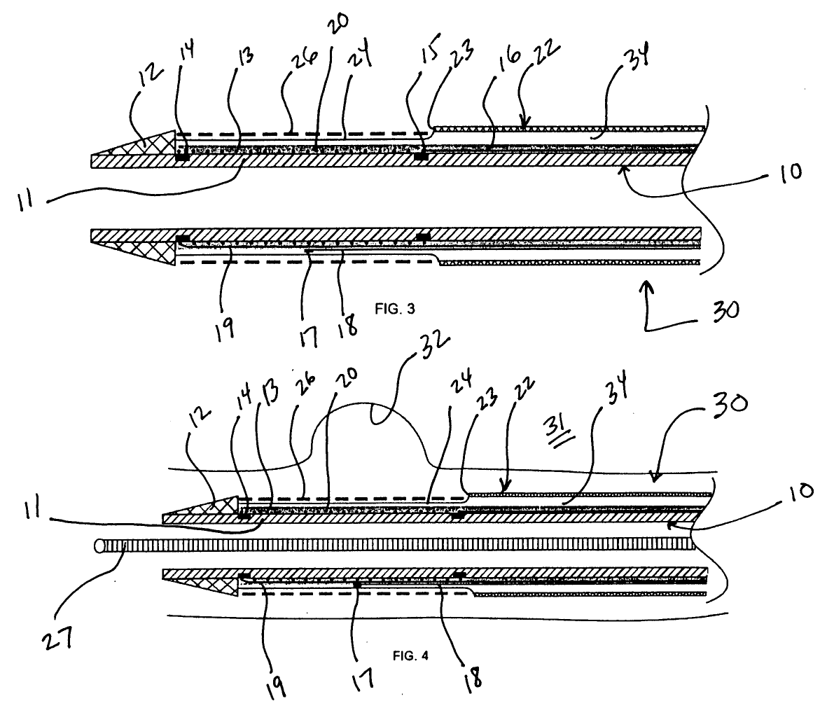

Turning to FIG. 2, an outer tube 22 is disposed over the inner tube 11. A distal end 23 of the outer tube 22 is connected to a balloon 24 which extends between a distal end 23 of the outer tube 22 and the distal tip 12 of the inner tube 10. An annular space 34 is provided between the inner tube and outer tube 22 for communication of inflation media or fluid to the balloon 24. Turning to FIG. 3, an expandable stent 26 may be positioned over the balloon 24. It is anticipated that the structure as shown in FIG. 2 (without the stent 26) will have some treatment applications, such as angioplasty or other related procedures.

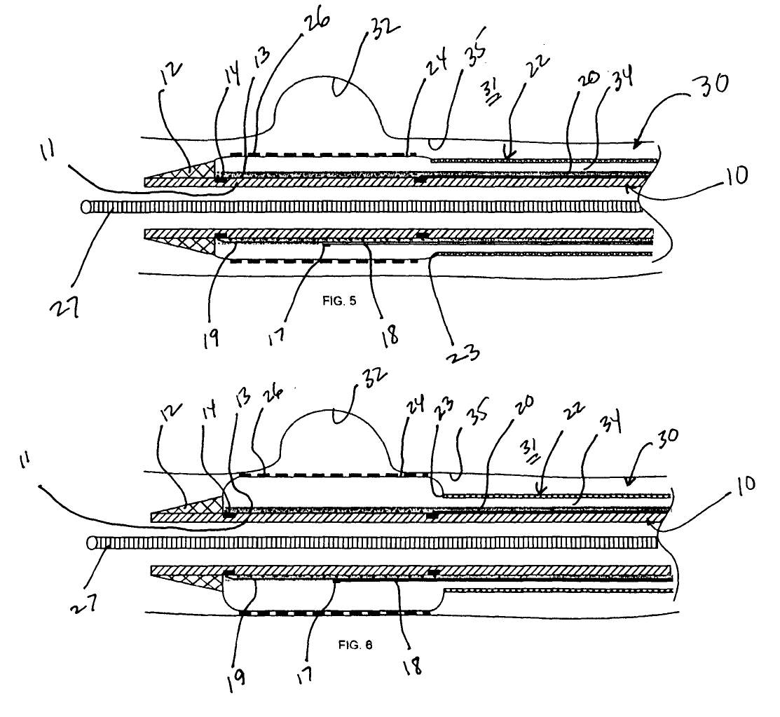

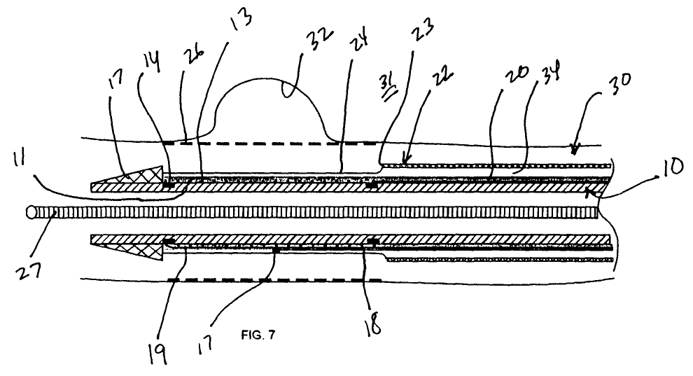

Turning to FIG. 4, the stent delivery system 30 is transported through the vasculature system 31 to the area to be treated 32 over a guidewire 27. The area to be treated 32, in this case, is an aneurism. The disclosed system 30 may also be used to treat stenoses and other ailments as well. As shown in FIG. 4, the stent 26 is in position and an indication as such can be transmitted to the physician by way of the distal and proximal radiopaque markers 14, 15. Turning to FIG. 5, with the stent 26 in position, inflation media or other fluid is transmitted through the annular space 34 to the interior of the balloon 24 to cause partial expansion thereof. Heat is transmitted to the balloon 24 and/or stent 26 by way of the heating element 13 which may be in the form of a coil or other structure. As the balloon 24 and stent 26 expands toward the arterial wall 35, additional inflation media and heat can be provided to the balloon until the stent 26 reaches its position against the arterial wall 35 as shown in FIG. 6. After the stent 26 has been expanded against the arterial wall 35 as shown in FIGS. 6 and 7, the balloon 24 is deflated to the position shown in FIG. 7 and the stent delivery device 30 is ready to be withdrawn from the vasculature system 31 over the guidewire 27 leaving the stent 26 behind.

Preferably, the stent 26 has a shape memory transition temperature above body temperature. One suitable material is nitinol. Using stent 26 with a shape memory transition temperature above body temperature allows it to be delivered to the diseased site without an outer sheath to constrain and prevent expansion of the stent 26. A packaging sheath may be used to prevent the stent from expanding prematurely during product storage and distribution. However, the packaging sheath can be removed prior to insertion of the stent delivery device into the vasculature system 31.

In addition, if a stent with a shaped memory transition temperature below body temperature is utilized, the stent may be cooled during stent delivery by transmitting a cooling medium through the annular space 34. It will also be noted that the distal tip 12 of the inner tube 10 and the distal end 23 of the outer tube 22 provides a recessed area for accommodating the stent 26 which protects the stent 26 as it travels up through the vasculature system. Again, this design feature may also eliminate the need for a protective sheath which can be detrimental to flexibility and trackability of the stent delivery system 30.

Instead of the heating element 13 and power line 16, heat can be supplied to the interior of the balloon 24 by transmitting heated inflation media through the annular space 34 to the interior of the balloon 24. Thus, the heating element 13 is optional. The balloon 24 should be fabricated from an elastomeric material. It will be noted that the balloon 24 is not necessarily used to expand the stent, rather it is heat supplied to the interior of the balloon by way of a heating element 13 or a heated medium transmitted through the annular space 34 that causes expansion of the stent in the preferred embodiment. The elastomeric material used to fabricate the balloon 24 ensures continual contact between the balloon 24 and stent 26 during deployment of the stent and effective transfer of heat between the balloon 24 and stent 26. Continual contact between the balloon 24 and stent 26 protects against the stent 26 from slipping during deployment of the stent 26 and therefore ensures accurate deployment thereof. The elastomeric balloon 24 will retract fully upon evacuation of inflation media through the annular space 34 thereby making withdrawal of the stent delivery system 30 safe even in a tortuous anatomy.

Further, passage of AC current through heating element 13 (i.e., an induction coil) made of wires of low resistivity such as of copper and silver will impart inductive heating. In induction heating, heat is generated within the stent 26 by the electromagnetic field generated by the induction coil 13. The stent 26 will heat up much faster and more uniform as compared to resistive heating.

For resistive heating, DC current is used and the heating element 13 would be made of wires with high resistivity such as platinum. In induction heating, AC current would be used and the heating element 13 would be made of wires with low resistivity such as silver and copper.

Although specific embodiments and methods have been described, workers skilled in the art will realize that changes may be made in form and detail without departing from the spirit and scope of this disclosure.

1. A stent delivery system comprising:
an inner tube comprising a proximal end and a distal end, the inner tube being disposed within an outer tube with an annular space disposed therebetween, the distal end of the inner tube further comprising a distal tip,
the outer tube comprising a proximal end and a distal end, the distal end of the outer tube being disposed proximally to the distal tip of the inner tube,
a heating element positioned around the inner tube proximal to the distal tip thereof,
the distal end of the outer tube being connected to a balloon which extends between the distal end of the outer tube and the distal tip of the inner tube, the balloon overlying the heating element.

2. The stent delivery system of claim 1 further comprising an expandable stent positioned around the balloon and disposed between the distal end of the outer tube and the distal tip of the inner tube.

3. The stent delivery system of claim 1 wherein the heating element comprises a coil.

4. The stent delivery system of claim 1 wherein the balloon is also connected to the distal tip of the inner tube.

5. The stent delivery system of claim 1 wherein the stent is crimped onto the balloon.

6. The stent delivery system of claim 1 wherein the stent comprises nitinol.

7. The stent delivery system of claim 1 wherein the stent is a self-expanding stent.

8. The stent delivery system of claim 1 wherein the balloon comprises an elastomeric material.

9. The stent delivery system of claim 1 wherein the distal tip of the inner tube is tapered.

10. The stent delivery system of claim 1 wherein the inner tube comprises a distal radiopaque marker disposed immediately proximal to the distal tip and a proximal radiopaque marker disposed immediately distal to the distal end of the outer tube.

11. The stent delivery system of claim 1 wherein the distal tip of the inner tube having a maximum outer diameter that is equal to or greater than a maximum outer diameter of the stent in an unexpanded form.

12. The stent delivery system of claim 1 wherein the heating element is connected to a power line and a return line that extends along the inner tube to the proximal end thereof.

13. The stent delivery system of claim 12 wherein the heating element, power line and return line are encased in a layer of insulating material.

14. A method of deploying an expandable stent in a vasculature system comprising:
providing a stent delivery system comprising
an inner tube comprising a proximal end and a distal end, the inner tube being disposed within an outer tube with an annular space disposed therebetween, the distal end of the inner tube further comprising a distal tip,
the outer tube comprising a proximal end and a distal end, the distal end of the outer tube being disposed proximally to the distal tip of the inner tube, the distal end of the outer tube being connected to a balloon which extends between the distal end of the outer tube and the distal tip of the inner tube,
a cylindrical expandable stent disposed around the balloon and disposed between the distal end of the outer tube and the distal tip of the inner tube,
the distal tip of the inner tube having a maximum outer diameter that is equal to or greater than a maximum outer diameter of the stent in an unexpanded form;
inserting the stent delivery system, with the stent in the unexpanded form, into the vasculature system and to a desired position;
heating the stent to expand the stent and causing it to adhere to the vasculature system at the desired position;
deflating the balloon; and
withdrawing the inner and outer tubes and balloon from the vasculature system.

15. The method of claim 14 wherein during the heating of the stent the balloon is heated and partially inflated.

16. The method of claim 14 wherein the stent is a self-expanding stent and during the inserting step, the balloon and stent are cooled.

17. The method of claim 14 wherein the cooling is performed using cool saline solution delivered through the annular space and into the balloon.

18. The method of claim 14 wherein the heating is performed using warm saline solution delivered through the annular space and into the balloon.

19. The method of claim 14 wherein the heating is performed using a heating element.

20. The method of claim 19 wherein the heating element comprises a coil connected to a power line that extends along the inner tube to the proximal end thereof and the current is supplied to the heating element through the power line.

21. The method of claim 19 wherein a thermocouple is disposed on the distal end of the inner tube and the method further comprises monitoring temperature during the heating step.

22. The method of claim 14 wherein the stent delivery system further comprises a protective sheath disposed over the stent and the method further comprises removing the protective sheath prior to the inserting step.

23. A stent delivery system comprising:
an inner tube comprising a proximal end and a distal end, the inner tube being disposed within an outer tube with an annular space disposed therebetween, the distal end of the inner tube further comprising a tapered distal tip, a heating element positioned on the distal end of the inner tube and proximal to the distal tip,
the outer tube comprising a proximal end and a distal end, the distal end of the outer tube being disposed proximally to the distal tip of the inner tube, the distal end of the outer tube being connected to a balloon which extends between the distal end of the outer tube and the distal tip of the inner tube, the balloon at least partially overlying the heating element,
the balloon also being connected to the distal tip of the inner tube, the balloon being disposed within and engaging a cylindrical expandable stent which overlies the balloon and is disposed between the distal end of the outer tube and the distal tip of the inner tube.

24. The stent delivery system of claim 23 wherein the stent is crimped onto the balloon.

25. The stent delivery system of claim 23 wherein the stent comprises nitinol.

26. The stent delivery system of claim 23 wherein the stent is a self-expanding stent.

27. A method of deploying an expandable stent in a vasculature system comprising:
providing a stent delivery system comprising
an inner tube comprising a proximal end and a distal end, the inner tube being disposed within an outer tube with an annular space disposed therebetween, the distal end of the inner tube further comprising a tapered distal tip,
a heating element being positioned on the distal end of the inner tube and proximal to the distal tip,
the outer tube comprising a proximal end and a distal end, the distal end of the outer tube being disposed proximally to the distal tip of the inner tube, the distal end of the outer tube being connected to a balloon which extends between the distal end of the outer tube and the distal tip of the inner tube, the balloon at least partially overlying the heating element, the balloon also being connected to the distal tip of the inner tube,
the balloon being disposed within and engaging a cylindrical expandable stent which overlies the balloon and is disposed between the distal end of the outer tube and the distal tip of the inner tube,
inserting the stent delivery system, with the stent in the unexpanded form, into the vasculature system and to a desired position;
heating the balloon and the stent by supplying current to the heating element while at least partially inflating the balloon to expand the stent and causing it to adhere to the vasculature system at the desired position.

28. The method of claim 27 further comprising:
deflating the balloon; and
withdrawing the inner and outer tubes and balloon from the vasculature system.

29. The method of claim 27 wherein the stent is a self-expanding stent and during the inserting step, the balloon and stent are cooled.

30. The method of claim 27 wherein the cooling is performed using cool saline solution delivered through the annular space and into the balloon.

31. The method of claim 27 wherein a thermocouple is disposed on the distal end of the inner tube and the method further comprises monitoring temperature during the inserting and heating steps.

32. The method of claim 27 wherein the stent delivery system further comprises a protective sheath disposed over the stent and the method further comprises removing the protective sheath prior to the inserting step.
